(19) United States
(12) Patent Application Publication
(10) Pub. No.: US 2024/0418676 A1
(43) Pub. Date: Dec. 19, 2024

(54) ELECTROCHEMICAL SENSOR FOR DETECTING AND CHARACTERIZING A BIOLOGICAL MATERIAL

(71) Applicant: Universiti Malaya, Kuala Lumpur (MY)

(72) Inventors: Vengadesh PERIASAMY, Kuala Lumpur (MY); Georgepeter Gnana Kumar, Tamil Nadu (IN)

(21) Appl. No.: 18/816,419

(22) Filed: Aug. 27, 2024

Related U.S. Application Data
(62) Division of application No. 17/615,055, filed on Nov. 29, 2021.

(30) Foreign Application Priority Data
Mar. 26, 2020 (MY) PI2020001607

(51) Int. Cl. G01N 27/48; G01N 33/68

(52) U.S. Cl. CPC G01N 27/48; G01N 33/6812

(57) ABSTRACT

Aspects of the present disclosure relate to an electrochemical sensor for detecting and characterising a biological material in a sample solution. The electrochemical sensor detects and characterises the biological material based on its electronic and electrochemical properties. The electrochemical sensor comprises three electrodes fabricated on a substrate, and a potentiostat.

### CROSS REFERENCE APPLICATION

This application is a Divisional Application of U.S. application Ser. No. 17/615,055, filed Nov. 29, 2021, which claims priority to, and the benefit of, PCT Application No. PCT/MY2020/050100, filed Oct. 8, 2020, which claims priority to, and the benefit of, MYPI2020001607 filed Mar. 26, 2020, the disclosures of which are hereby incorporated by reference in their entireties.

### FIELD OF INVENTION

The present invention relates to an electrochemical sensor for detecting and characterizing a biological material. More particularly, the present invention relates to a printed circuit board based electrochemical sensor for detecting and characterizing a biological material.

### BACKGROUND OF THE INVENTION

An electrochemical sensor allows the transformation of a biological or chemical signal into an electrical signal that can be useful for detection and characterisation of a biological material. The electrochemical sensor is typically composed of a sensing platform which may include a plurality of electrodes connected to an electronic hardware that controls the operation of the electrochemical sensor. However, this conventional arrangement requires a bulky and elaborate equipment set-up with a large amount of sample required for testing.

In view of this, there is a need to provide a miniaturised electrochemical sensor that facilitates the detection and analysis of the biological material. One example of the miniaturised electrochemical sensor is disclosed in German Patent Publication No. DE10332804 A1 which provides a biosensor comprising of a plurality of electrodes arranged on a surface of a printed circuit board to detect electrochemical reaction between a target biomolecule in a sample and a biomolecule probe.

However, such devices may have certain limitations in determining and analysing the target biomolecule as the devices rely on a method of sample preparation and measuring a redox reaction that resulted from the interaction between the sample and the probe. This restricts the type of biological material that can be detected by such electrochemical sensor.

Therefore, there is a need for a sensor and method that addresses the abovementioned drawbacks whereby such sensor and method would be able to perform detection and characterisation on various types of biological material.

### SUMMARY OF INVENTION

In one aspect of the present invention, an electrochemical sensor for detecting and characterising a biological material is provided. The electrochemical sensor comprises a working electrode (111), a counter electrode (112), a reference electrode (113), and a potentiostat connected to the working electrode (111), the counter electrode (112) and the reference electrode (113). The working electrode (111), the counter electrode (112) and the reference electrode (113) are fabricated on a printed circuit board (100). The potentiostat is configured to obtain a current-potential profile of the biological material.

Preferably, the working electrode (111) is connected to a first connecting pad (121) via a first conducting track (131), the counter electrode (112) is connected to a second connecting pad (122) via a second conducting track (132), and the reference electrode (113) is connected to a third connecting pad (123) via a third conducting track (133). All of the conducting tracks (131, 132, 133) are suitably covered by epoxy solder mask.

Preferably, the working electrode (111), the counter electrode (112) and the reference electrode (113) are made out of a same metal material.

Preferably, the working electrode (111), the counter electrode (112) and the reference electrode (113) are arranged spaced apart from each other.

In another aspect of the present invention, a method for characterizing a biological material is provided. The method is characterised by the steps of depositing a sample of the biological material onto the working electrode (111), the counter electrode (112), and the reference electrode (113); supplying a potential through the working electrode (111) at a consecutive range under a specific scan rate; measuring a current response as the supplied potential is swept linearly in time; and establishing a current-potential or I-V profile of the biological material based on the measured current response.

Preferably, the step of establishing the current-potential profile includes obtaining a current density of the working electrode via a linear sweep voltammogram.

In yet another aspect of the present invention, a method for detecting an unknown biological material in a sample is provided. The method is characterised by the steps of depositing the sample onto the working electrode (111), the counter electrode (112), and the reference electrode (113); supplying a potential through the working electrode (111) at a consecutive range under a specific scan rate; measuring a current response as the supplied potential is swept linearly in time; establishing a current-potential or I-V profile of the unknown biological material based on the measured current response; and comparing the I-V profile with a database having a list of known biological materials and its respective I-V profiles to identify the unknown biological material.

Preferably, the step of establishing the current-potential profile includes obtaining a current density over the current-surface area of the working electrode; and obtaining a linear sweep voltammogram based on the current density.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 shows a printed circuit board (100) of an electrochemical sensor according to an embodiment of the present invention.

FIGS. 2(a-b) show exemplary linear sweep voltammograms for sterile water, I-arginine, serine, and alanine.

### DESCRIPTION OF THE PREFERRED EMBODIMENT

A preferred embodiment of the present invention will be described hereinbelow with reference to the accompanying drawings. In the following description, well-known functions or constructions are not described in detail since they would obscure the description with unnecessary detail.

The present invention relates to an electrochemical sensor for detecting and characterising a biological material in a sample solution. The biological sample may be obtained from a human, animal or plant subject and may be provided in a form of biological solution. Examples of the biological solution include blood, sweat, tears, serum, and saliva from which biomolecules such as proteins, amino acids, or nucleic acids can be identified. The electrochemical sensor detects and characterises the biological material based on its electronic and electrochemical properties. The electrochemical sensor comprises three electrodes fabricated on a substrate, and a potentiostat.

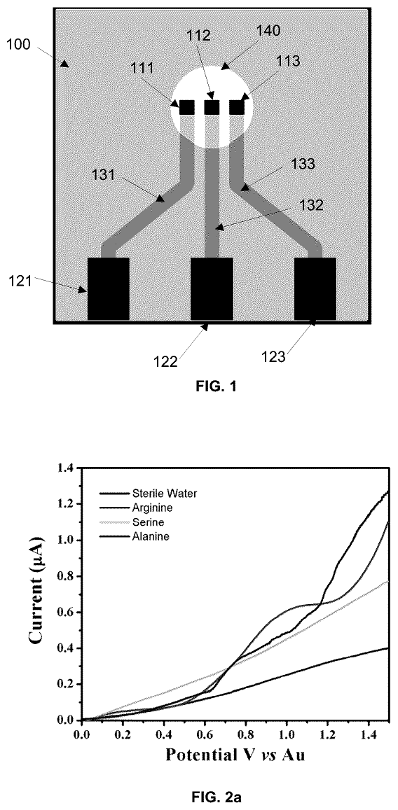

Referring to FIG. 1, there is shown a layout of the electrodes fabricated on a printed circuit board or PCB (100). The PCB (100) includes of a working electrode (111), a counter electrode (112), a reference electrode (113), and three connecting pads (121, 122, 123). The working electrode (111) is electrically connected to a first connecting pad (121) through a first conducting track (131), the counter electrode (112) is electrically connected to a second connecting pad (122) through a second conducting track (132), and the reference electrode (113) is electrically connected to a third connecting pad (123) through a third conducting track (133). Preferably, all of the conducting tracks (131, 132, 133) are covered by epoxy solder mask so as to prevent the conducting tracks from being exposed to the biological material deposited on the electrodes (111, 112, 113).

The working electrode (111), the counter electrode (112), and the reference electrode (113) are suitably made out of at least one or combination of conducting materials selected from the group consisting of gold (Au), silver (Ag), titanium (Ti), platinum (Pt), iridium (Ir), and the like of electrode materials. Moreover, all electrodes are preferably made out of the same metal material so as to avoid electrode polarization at an electrode-electrolyte interface. The electrode polarization may cause large potential drop at the electrode-electrolyte interface, and thereby, causing lower sensitivity. In addition to that, the use of different metal material for each electrode may cause the electrodes to exhibit different junction properties that will effectively mask the possible Schottky-like junction from the biological material-metal junctions.

The working electrode (111), the counter electrode (112) and the reference electrode (113) are arranged spaced apart from each other. Preferably, the electrodes are fabricated in a parallel configuration, wherein the reference electrode and the counter electrode are separated by a gap of approximately 0.5 mm and the counter electrode and the working electrode are separated by a gap of approximately 0.5 mm. Such arrangement of the electrodes is to maximize charge injection across the biological material-metal junction, wherein the electrodes are fabricated to be within a sample loading area (140) where the surface area is deposited with the sample solution. Thus, a non-symmetrical electronic response is observed from an asymmetrical junction.

The working electrode (111) is where sensing of current response is carried out during a stimulation of the sample solution. The current response can be normalized by obtaining a current density over the current-surface area of the working electrode (111) to allow comparison with other results. The counter electrode (112) is configured to pass all the current needed to balance the current observed at the working electrode (111). The reference electrode (113) is configured to act as a point of reference for potential measurements by holding a constant potential at low current density.

The working electrode (111), the counter electrode (112) and the reference electrode (113) are connected to the potentiostat via the respective connecting pads (121, 122, 123). The potentiostat is configured to obtain a current-potential profile of the biological material. In particular, the potentiostat is configured to control and maintain a potential of the working electrode (111) at a constant level with respect to the reference electrode (113) by adjusting the current at the counter electrode (112). It would be apparent by a person skilled in the art that the potentiostat may be a single integrated unit or a configuration of multiple set of modules, components, or equipment for controlling and maintaining the potential of the working electrode (111) at a constant level with respect to the reference electrode (113) by adjusting the current at the counter electrode (112). The potentiostat may also obtain a current density by normalizing the current with the surface area of the working electrode (111).

A method for characterizing a biological material in a sample solution is provided hereinbelow. Initially, a trace amount of the sample solution is deposited onto the sample loading area (140), wherein the sample solution is deposited so as to sufficiently form an electrical contact with the working electrode (111), the counter electrode (112), and the reference electrode (113).

Thereon, a potential is supplied through the working electrode (111) at a consecutive range under a specific scan rate. As the potential is swept linearly in time, a current response is generated and measured at the working electrode (111). The current response may be normalized by obtaining a current density over the current-surface area of the electrode. In other words, the current density is obtained by normalizing the current response with the surface area of the working electrode (111).

A current-potential or I-V profile of the biological material is established and determined based on the measured current response or the normalized current response, wherein the I-V profile indicates a unique electrochemical signature of the biological material. In other words, the biological material is characterised by a linear sweep voltammogram obtained from the sample solution.

A method for detecting an unknown biological material in a sample solution is provided hereinbelow. Initially, a trace amount of the sample solution is deposited onto the sample loading area (140), wherein the sample solution is deposited so as to sufficiently form an electrical contact with the working electrode (111), the counter electrode (112), and the reference electrode (113).

Thereon, a potential is supplied through the working electrode (111) at a consecutive range under a specific scan rate. As the potential is swept linearly in time, a current response is generated and measured at the working electrode (111). The current response may be normalized by obtaining a current density over the current-surface area of the electrode. In other words, the current density is obtained by normalizing current with the surface area of the working electrode (111).

A current-potential or I-V profile of the biological material is established and determined based on the measured current response or the normalized current response. Thus, a linear sweep voltammogram of the unknown biological material is obtained from the measured current response or the normalized current response.

The characteristics of the linear sweep voltammogram are then compared with a database having a list of known biological materials and its respective linear sweep voltammograms. If the characteristics of the linear sweep voltammogram of the unknown biological material match one of the linear sweep voltammograms in the database, the unknown biological material is detected and identified as the particular biological material having the matched linear sweep voltammogram stored in the database.

Hereinafter, the present invention is further illustrated by the following examples. The examples used herein are intended merely to facilitate an understanding of ways in which the embodiments herein may be practised and to further enable those of skill in the art to practise the embodiments herein.

### Example

### Fabrication of the Printed Circuit Board

A printed circuit board or PCB based on the layout is as shown in FIG. 1. In particular, the PCB was a single-sided FR4 1.6 mm designed with three electrodes and three connecting pads, wherein each electrode was connected to one of the connecting pads by a copper track with a thickness of approximately 36 μm. The electrodes and connecting pads were electroplated nickel-gold plates having a nickel thickness layer of approximately 4 to 5 μm and a nickel thickness layer of approximately 0.049 to 0.052 μm. The copper tracks were covered by epoxy solder mask to allow only the electrodes and the connecting pads being exposed. Prior to the experiment, the PCB was pre-treated by sonicating in acetone, rinsing using deionized water, sonicating in ethanol, rinsing using deionized water, and drying using Nitrogen gas.

### Experimental Setup of the Electrochemical Sensor

The PCB was connected to a potentiostat via the connecting pads. The potentiostat was configured to supply a potential at a range of 0 to 1.5 V under a scan rate of 20 mV s⁻¹.

### Characterisation of Amino Acids

10 μL of sterile water was deposited onto the sample loading area of the PCB. Thereon, a potential is supplied through the working electrode (113) at a consecutive range of 0 to 1.5 V under a scan rate of 20 mV s⁻¹. As the potential swept linearly in time, a current response generated at the working electrode (113) was measured and recorded. In addition to that, the current response was normalized to obtain a current density, wherein the surface area of the working electrode was determined to be 0.049 mm². The process was repeated for I-arginine, serine, and alanine having a concentration of 100 ng μL⁻¹.

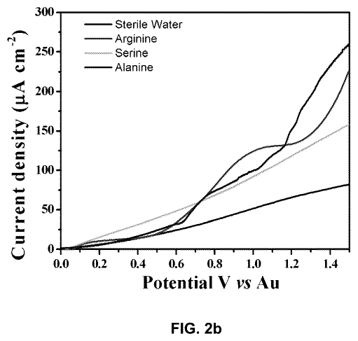

FIGS. 2(a-b) show the linear sweep voltammograms plotted based on the measured current response and current density for sterile water, I-arginine, serine, and alanine. It was observed that the characteristics of the linear sweep voltammograms for sterile water, I-arginine, serine, and alanine differ from one to another.

While embodiments of the invention have been illustrated and described, it is not intended that these embodiments illustrate and describe all possible forms of the invention. Rather, the words used in the specifications are words of description rather than limitation and various changes may be made without departing from the scope of the invention.

1. A method for characterizing a biological material is characterised by the steps of:
a) depositing a sample of the biological material onto the working electrode, the counter electrode, and the reference electrode;
b) supplying a potential through the working electrode at a consecutive range under a specific scan rate;
c) measuring a current response as the supplied potential is swept linearly in time; and
d) establishing a current-potential or I-V profile of the biological material based on the measured current response.

2. The method as claimed in claim 1, wherein the step of establishing the current-potential profile includes obtaining a linear sweep voltammogram based on a current density, wherein the current density is obtained by normalizing the current response with a surface area of the working electrode.

3. A method for detecting an unknown biological material in a sample is characterised by the steps of:
a) depositing the sample onto the reference electrode, the counter electrode, and the working electrode;
b) supplying a potential through the working electrode at a consecutive range under a specific scan rate;
c) measuring a current response as the supplied potential is swept linearly in time;
d) establishing a current-potential or I-V profile of the unknown biological material based on the measured current response; and
e) comparing the I-V profile with a database having a list of known biological materials and its respective I-V profiles to identify the unknown biological material.

4. The method as claimed in claim 3, wherein the step of establishing the current-potential profile includes obtaining a linear sweep voltammogram based on a current density, wherein the current density is obtained by normalizing the current response with a surface area of the working electrode.
